(19) United States
(12) Patent Application Publication
(10) Pub. No.: US 2005/0283111 A1
(43) Pub. Date: Dec. 22, 2005

(54) Catheter assembly with degradable material

(76) Inventor: Dan Maurice, Monument Beach, MA (US)

Correspondence Address: TECHNOPROP COLTON, L.L.C., P O BOX 567685, ATLANTA, GA, 311567685, US

(21) Appl. No.: 11/158,222

(22) Filed: Jun. 21, 2005

Related U.S. Application Data
(60) Provisional application No. 60/581,984, filed on Jun. 22, 2004.

(51) Int. Cl. A61M 3/00

(52) U.S. Cl. 604/43

(57) ABSTRACT

A catheter assembly having a first catheter, a second catheter, a degradable material between at least a portion of the first and second catheters for releasably joining at least a portion of the first catheter and the second catheter to each other.

### STATEMENT OF RELATED APPLICATIONS

This application is a Nonprovisional patent application claiming priority on U.S. Provisional Patent Application No. 60581984 entitled “Catheter Assembly With Degradable Material” having a filing date of 22 Jun. 2004.

### BACKGROUND OF THE INVENTION

1. Technical Field

This invention relates generally to the field of catheters for use in applications that require blood, fluids, medicated solutions, and other solutions to be removed from and/or introduced into a person. More particularly, this invention relates to catheters having at least two independent lumens that initially are maintained together and that can be separated at their distal ends.

2. Prior Art

Catheters for the introduction or removal of fluids throughout the body have been well characterized in the prior art for both single and multiple lumen catheters designs. Such types of catheters are used in hemodialysis, which involves the temporary removal of blood from a patient for the purpose of extracting toxins from the blood and then returning of the cleansed blood to the same patient. In conventional catheter insertion procedures and in accordance with the Seldinger technique, a needle is introduced subcutaneously into a localized blood vessel, which is confirmed by return blood flow through the needle into a syringe. Then, a guide wire is introduced through the needle and into the vessel leaving a portion of the guide wire outside the body.

One of the more common techniques to facilitate catheter placement through the subcutaneous tunnel and into the vessel is to place the catheter over the guide wire and directly into the vessel. The catheter follows the path created by the guide wire until the desired location is reached. At that point, the guide wire is removed from the body, leaving the catheter in place. This technique has limitations for devices significantly larger than the guide wire diameter. Typically, for devices significantly larger than the guide wire diameter, a series of dilators are used to gradually enlarge the subcutaneous tunnel and the opening in the vessel until the opening requirements meet the needs of the practitioner.

Single and multiple lumen catheters that have a solitary or staggered tip design similar to and described in the U.S. Pat. No. 4,643,711 are ideal for this known method of insertion. However, catheters that terminate in a split-tip configuration are difficult to insert using this standard technique as well as other preferred techniques. Typically, the physician will have to “weave” the guide wire through a myriad of holes located at the distal ends of either or both of the catheters of a split catheter structure in order to keep the previously separated distal segments together. However, even after this preventative separation measure is performed, each of the distal catheter segments have a propensity to deflect, “bow-up” and “hang-up” on the guide wire within the vessel making the procedure more difficult, time consuming, and exposing the patient to needless risk as well as vessel damage.

Thus, it can be seen that a need exists for a method to improve the insertion of catheters into patients. Such a method could allow a catheter structure to be inserted as a single piece, which would reduce the need to “weave” the catheter, and then allow the multiple catheter ends to separate within the body. Such a method could allow for the catheter to be inserted using standard techniques. It is to these needs, among others, that this invention is directed.

### BRIEF SUMMARY OF THE INVENTION

The present invention comprises a single piece catheter assembly having multiple catheters (and lumens), with the catheters releasably joined together at their distal end by a degradable material. The distance between the multiple, but generally two, lumens at distal ends can be varied by the degradation of the degradable material therebetween.

The catheter assembly of the present invention is intended for use in applications in which bodily fluids, medicated solutions, and other solutions are introduced into and/or removed from the body. For example, in the illustrative case of hemodialysis treatment, one embodiment of the invention is useful for the removal of blood from a patient for purification and for reintroducing the purified blood into the patient through the same blood vessel. In such applications, the catheter assembly can be used in many blood vessels including the femoral blood vessels, subclavian blood vessels or other blood vessels.

Typically, the multiple catheters, each of which comprises a lumen, of a preferred catheter assembly are generally parallel to each other at and near their distal ends and are connected to a common hub at their proximal ends. As the multiple catheters are for the most part independent from each other after extending beyond the common hub, it may be difficult to insert the multiple lumen catheters into the patient's body. A single piece catheter assembly having multiple catheters comprising a degradable material disposed between and releasably holding the multiple catheters together is disclosed. When the degradable material comes in contact with a fluid, e.g. a bodily fluid such as blood within the vessel, the degradable material degrades, releasing the multiple catheters and thus the multiple lumens from each other.

In one embodiment, the degradable material is selected so that it effectively allows for the time release of the tips of the catheters. Specifically, the degradable material may be selected so that it degrades in a specific period of time. Thus, one of ordinary skill in the art can select a degradable material that allows for a controlled separation of the catheters after the catheter assembly has been installed in a patient.

In operation and use, the practitioner places the catheter assembly into the patient as a single unit. The flow of fluid, such as the surrounding blood, causes the degradable material to degrade, thereby releasing the distal ends of the catheters from each other. As a practitioner may insert the catheter assembly into a patient as a single unit, the patient is at less risk and the catheter assembly may be placed more easily in the patient. Specifically, the ability of the catheter assembly to be installed into a patient as a single unit reduces the need for the use of the “weave” technique that is generally needed for many multiple catheter installation. As such the catheter assembly is safer and easier to install.

These features, other features and advantages of the present invention will become more apparent to those of ordinary skill in the relevant art when the following detailed description of the preferred embodiments is read in conjunction with the appended drawings in which like reference numerals represent like components throughout the several views.

### BRIEF DESCRIPTION OF THE DRAWINGS

FIG. 1 is a perspective view of one illustrative embodiment of the present invention.

FIG. 2 is a cross section along line 2′-2′ of the embodiment shown in FIG. 1.

FIG. 3 is a sectional view of a patient's body with the embodiment shown in FIG. 1 placed therein.

FIG. 4 is perspective view of a second illustrative embodiment of the present invention.

FIGS. 5A-C are enlarged cross-sectional views of multiple lumen catheter assemblies in accordance with additional embodiments of the present invention.

### DETAILED DESCRIPTION OF THE PREFERRED EMBODIMENTS

Illustrative embodiments of the present invention include a multiple lumen catheter assembly 10 that has at least two joinable catheters 12, 14. The distance between two of the at least two catheters 12, 14 at a distal section or end 20 of the catheter assembly 10 may be varied by the degradation of degradable material 40 therebetween. While the invention is described herein in conjunction with the preferred and illustrative embodiments, it will be understood that the invention is not limited to these embodiments.

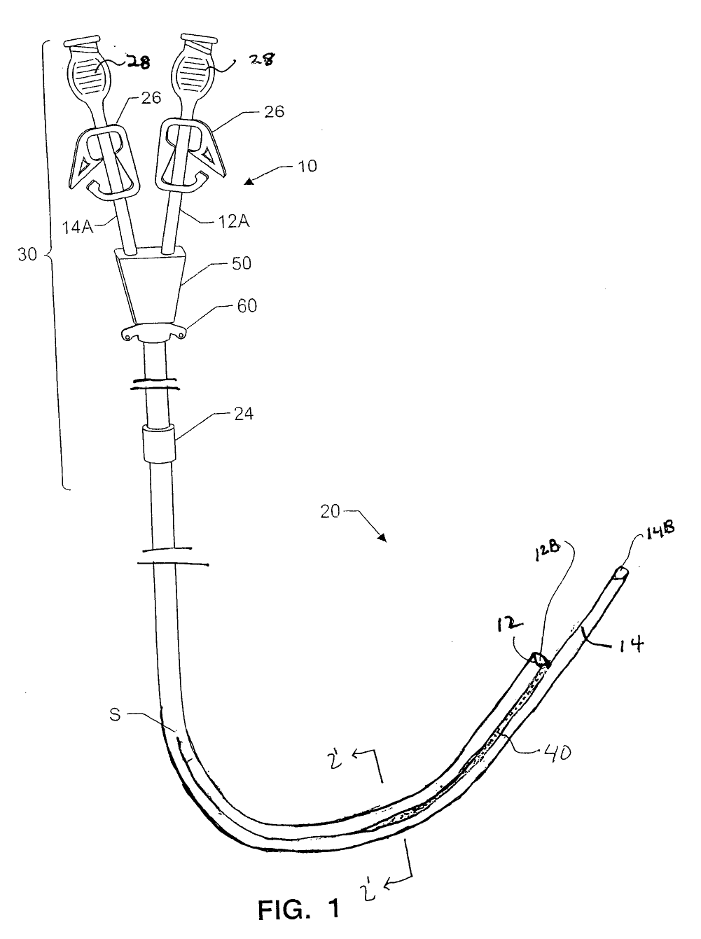

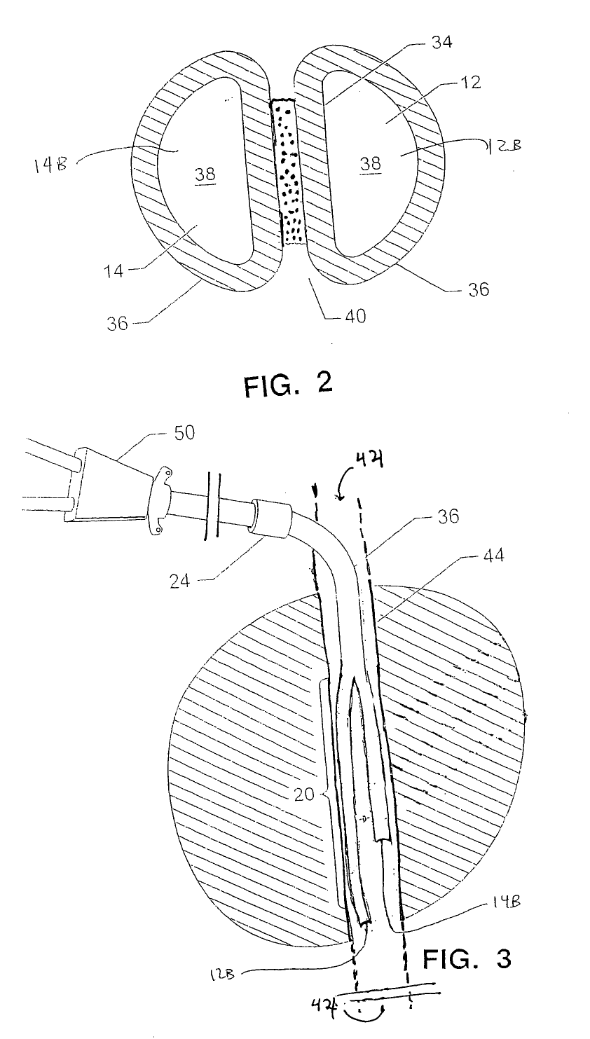

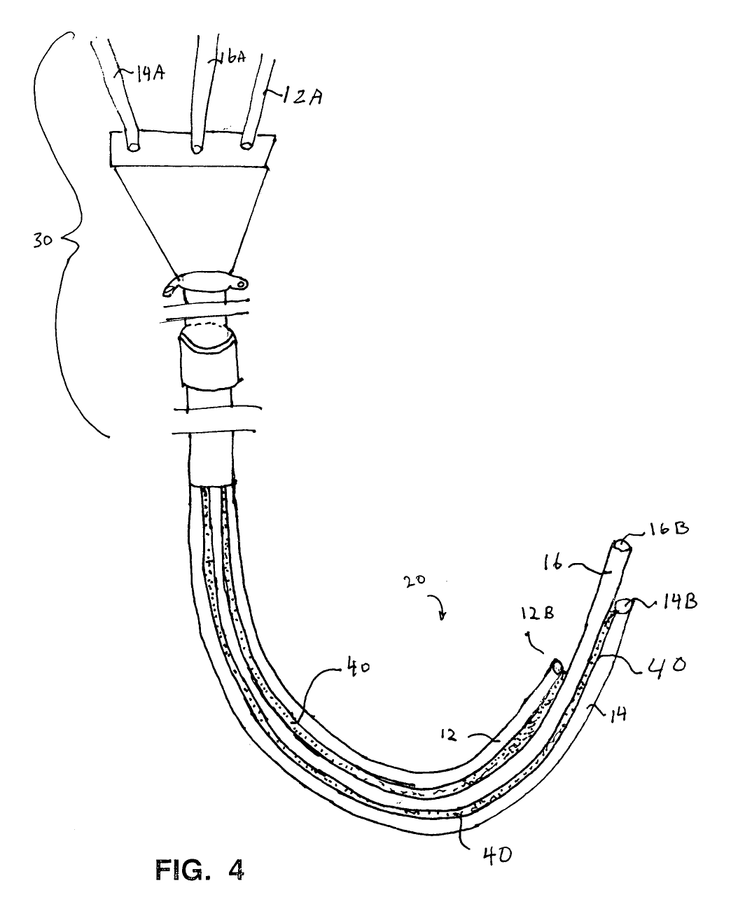

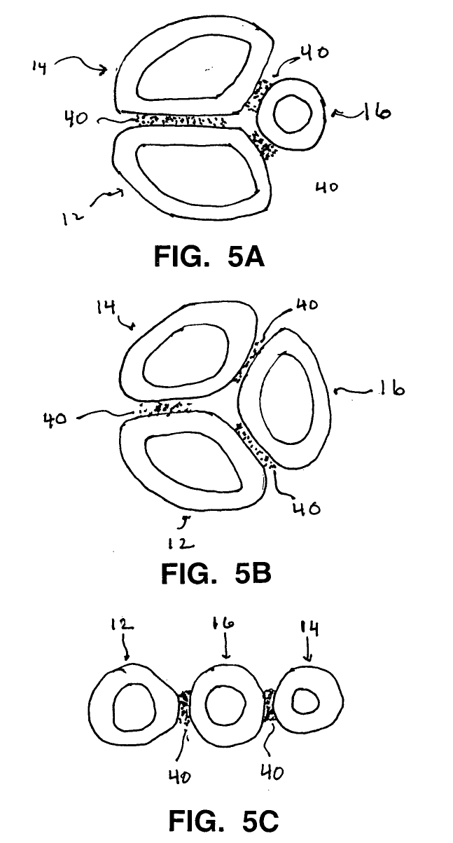

Catheter assembly 10 comprising multiple catheters 12, 14 may be constructed in accordance with the illustrative embodiments of the present invention as shown in FIGS. 1-4. FIG. 1 is a perspective view of catheter assembly 10 showing the general structure of one illustrative embodiment with catheters 12, 14 constructed with a degradable material 40 in an unstressed configuration. FIG. 2 is a cross-sectional view of a distal section 20 of the embodiment shown in FIG. 1 showing generally the time releasable distal ends of catheters 12, 14 constructed with degradable material 40 joining the opposing walls of catheters 12, 14. FIG. 3 is a schematic view of the embodiment shown in FIG. 1 within the body of a patient illustrating the degrading of degradable material 40 and the releasing of the distal ends of catheters 12, 14 from each other. FIG. 4 is perspective view of a second illustrative embodiment of the present invention. FIGS. 5A-C are enlarged cross-sectional views of multiple lumen catheter assemblies in accordance with additional embodiments of the present invention.

Catheter assembly 10 primarily is intended for use in applications in which bodily fluids, medicated solutions, and other solutions are introduced into and removed from the body. For example, in the illustrative case of hemodialysis treatment, one embodiment of the invention is useful for the removal of blood from a blood vessel for purification and for reintroducing the purified blood into the patient through the same blood vessel. In such applications, catheter assembly 10 can be used in many blood vessels including the femoral blood vessels, subclavian blood vessels or other blood vessels.

Referring now to FIG. 1, catheter assembly 10 has two ends with distinctive functions. More particularly, distal end 20 of catheter assembly 10 is adapted to extend within blood vessel 44 of a patient and proximal end 30, opposite distal end 20, of catheter assembly 10 is adapted to remain outside the body of a patient for cooperating with a means for introducing into, withdrawing from, and/or exchanging fluids from a blood vessel 44. Preferably, catheters 12, 14 are approximately parallel to one another and are long enough to extend though a subcutaneous tunnel from an exit site to catheterized blood vessel 44. Between the non-degradable catheters 12, 14 walls is degradable material 40 that secures the distal ends of catheters 12, 14 together.

This representative catheter assembly 10 comprises two catheters 12, 14 extending from common hub 50. Generally catheters 12, 14 are part of a single unit having two catheters which split apart from each other at location S. Often, catheters 12, 14 have a “D” shape with the flat part of the “D”-shaped cross section forming facing, opposing walls. Degradable material 40 is manufactured or placed between the non-degradable opposing walls of catheters 12, 14 and preferably has a time releasable characteristic. Preferably, the walls of catheters 12, 14 are close but are not in contact with each other.

Catheters 12, 14 are formed from independent tubular structures capable of carrying fluids. Each tubular structure has a relatively constant wall thickness and flow cross-section across its length. Each of catheters 12, 14 is preferably constructed of a material of a sufficient rigidity to maintain its general shape under normal usage, including instances when negative pressure is applied to catheter assembly 10 (for example when aspirating blood from blood vessel 44) or when positive pressure is applied to the catheter assembly 10 (for example when introducing blood to blood vessel 44). Also preferably, as catheter assembly 10 can be within the patient for a significant period of time, each catheter 12, 14 preferably is sufficiently flexible to avoid discomfort, trauma, or stenosis to the patient.

As shown in FIG. 2, catheters 12, 14 of catheter assembly 10 are releasably joined to each other by degradable material 40 at at least one position along the region between split S and distal end 20. Preferably, catheters 12, 14 are time releasable generally along the facing flat walls of catheters 12, 14 through joining by degradable material 40. Degradable material 40 may be degraded by solution contact and flow, for example, by contact with blood flowing around surrounding catheters 12, 14. As degradable material 40 degrades releasing catheters 12, 14 form each other, the distance between the distal tips of catheters 12, 14 will increase distance D, thus separating the end of the lumens 12B, 14B of catheters 12, 14 from each other along the distance from split S to the distal ends 20.

Referring now to FIG. 3, catheters 12, 14 may be separate or independent from each other starting at many different points and thus achieve a degree of separation within the blood vessel 44 of the patient. As shown in the illustrative structure of FIG. 1, catheters 12, 14 shown are part of a generally unitary double catheter structure for approximately half their total length, or until split S. Split S may be at any preselected distance from connecting hub 50, but preferably should be at a maximum distance so as to allow catheters 12, 14 to be separate and independent from each other for a medically appropriate distance. For example, the medically appropriate distance for a hemodialysis catheter can be the maximum desirable distance for use in a human being in the 90%+size range. After degradable material 40 has been degraded, the catheter assembly will remain joined above split S. Split S may be manufactured at different places along catheter assembly 10 so as to provide an appropriately sized catheter assembly 10. Further, a series or system of different sized catheter assemblies 10 can be made for use in various patients of different sizes or having different sized body cavities. Similarly, the catheter assembly 10 can be manufactured in way to allow the sizing of the lengths of catheter 12, 14 such as by allowing the cutting or breaking off at set or selected lengths along the distal ends of catheter 12, 14.

As shown, the independence of catheters 12, 14 relative to each other can confer several benefits to catheter assembly 10. The distinct surface walls 34 of each of catheters 12, 14, shown in FIG. 2, can add structural stability to catheter assembly 10. Further, as catheters 12, 14 have individual walls, it is possible to have separate lumens 12B, 14B and thus create the split tipped structure of catheter assembly 10. Importantly, after the distal ends of catheters 12, 14 are in separation, the distal ends of catheters 12, 14 are in a free-floating configuration in the same blood vessel 44 and are able to provide higher flow rates with lower pressures and with less occlusion, as is known in the art.

The time releasable joinable tip structure of catheter assembly 10 allows catheters 12, 14 to be spaced apart from one another without compromising the structure of each of catheters 12, 14. Specifically, after degradable material 40 has been degraded, distance D is created between the tips of catheters 12, 14, which is essential for good fluid flow. For example, in a hemodialysis application, separation distance D may help to prevent mixing of cleansed blood returning through one lumen 12B, 14B with the blood being removed from the blood vessel 44 through another one of lumens 12B, 14B. Generally, a larger separation distance D allows for less mixing of fluids being returned with fluids being removed.

Referring now to FIG. 4, another embodiment of this invention includes a catheter assembly 10 having more than two catheters. For example, catheter assembly 10 includes three parallel catheters 12, 14, 16. At the proximal end of catheter assembly 10, the three lumens 12, 14, 16 open up so as to form three internal, separate passageways which communicate with the proximal ends of the catheters 12, 14, 16. As a result, when inserted and in use, blood can be removed and returned in a closed loop with a hemodialysis machine using the two of the catheters 12, 14 and third catheter 16 is available for intravenous infusion of liquid medicaments. To prevent the entrance of blood into catheter 16, between uses, that is between infusions of liquid mendicants in this example, the third catheter may be filled with a relatively small volume of heparin or may be occupied by a cylindrical solid similar to a guide wire.

Referring now to FIGS. 5A-5C, it is contemplated that the degradable material 40 may between at least two of the catheters 12, 14, 16. FIGS. 5A and 5B show embodiments in which degradable material 40 resides between the three catheters 12, 14, 16. FIG. 5C shows an embodiment in which the degradable material 40 resides between first and second catheters 12 and 14 and between the second and third catheters 14 and 16. Other variations of catheters assemblies 10 can exist that may be constructed with degradable material 40 between two or more of catheters 12, 14, 16 with the catheters (or lumens therein) having further subdivisions, and/or with four or more catheters.

In operation and use, catheter assembly 10 is supplied to a practitioner in an unreleased configuration, meaning that catheters 12, 14, (and/or 16) are connected to each other by undegraded degradable material 40. The practitioner then places catheter assembly 10 into the patient as a single unit and the flow of fluid around catheters 12, 14 (and/or 16) causes degradable material to degrade, thereby releasing catheters 12, 14 (and/or 16) from each other. As the practitioner may insert catheter assembly 10 into a patent as a single unit, the patent is at less risk. Specifically, the ability of catheter assembly 10 to be installed into a patient as a single unit decreases the clinical difficulties associated with the “weave” technique generally needed for the catheter installation.

Degradable material 40 may be any material sufficiently adheres to the external walls of catheters 12, 14, (and/or 16) so as to maintain the catheters 12, 14, (and/or 16) together and degrades when exposed to solutions and bodily fluids such as blood fluid or plasma to allow the separation of catheters 12, 14, (and/or 16), and which is non-toxic or acceptable toxicity to patients. Preferably, degradable material 40 is a material selected from the material such as bioerodible materials, bioabsorable materials, biodegradable materials, and combinations thereof. Suitable synthetic materials include polyglycolic acid (PGA), poly-gylcolic and poly-L-lactic acid copolymers, polycaprolactones (for example as disclosed in U.S. Pat. No. 4,702,917 to Schnidler), polyhydroxybutyrate and hydroxyvalerate copolymers, polycarbonates, poly-L-lactides, polydioxanones, and polyanhydrides. Other naturally occurring and synthetic materials that are suitable include collagen, gelatin, elastin, laminin, fribrinogen, fribronectin, vitronectin, dextrose, sucrose, glucose, and fructose. One of ordinary skill in the art can select a degradable material suitable for degradable material 40 without undue experimentation.

In one embodiment, degradable material 40 is selected so that it effectively allows for the time-released separation of the distal tips of catheters 12, 14 (and/or 16) within a specific period of time. More particularly, the catheters 12, 14, (and/or 16) are released after the degradable material has been degraded, which degrades over a period of time. Thus, the release of the catheters 12, 14 (and/or 16) may be controlled by selecting an appropriate degradable material 40.

For example, it has been found that the use of a certain synthetic and natural polymer allows catheters 12, 14 (and/or 16) to remain together for a period of approximately 6 months in storage, yet release within minutes upon contact with bodily fluids. Further, it has been found that the use of dextrose allows catheters 12, 14 (and/or 16) to remain together for a period of hours. Thus, one of ordinary skill in the can select degradable material 40 that allows for the controlled separation of catheters 12, 14 (and/or 16) after catheter assembly 10 has been installed in a patient.

Each of catheters 12, 14 (and/or 16) can be manufactured to have a length and width useful for catheterization of an intended blood vessel 44 and/or other application. As shown, catheters 12, 14 (and/or 16) can be longitudinally spaced by a sufficient distance so as to essentially prevent or reduce the chance for the mixing of the blood to be removed and the blood to be reintroduced. This is shown illustratively in FIG. 3, where the openings of lumens 12B, 14B are longitudinally separated from each other. The optimal longitudinal distance between the openings will vary for different applications and with the patient that will be catheterized. One of ordinary skill in the art can manufacture catheter assembly 10 to desired specifications without undue experimentation. Further, the catheter assembly 10 can be manufactured in such a manner so as to allow the practitioner to size catheters 12, 14, (and/or 16) for a particular patient.

The illustrative catheter embodiment disclosed herein may be used for the intake or removal of blood from a blood vessel to be detoxified and for the return of such detoxified blood into that same blood vessel. A suitable hemodialysis machine (not shown) may be attached in fluid communication with catheter assembly 10 using adaptors or luer locks 28 (shown in FIG. 1). The catheter assembly 10 may also be used to deliver and remove fluids from the patient. Further, in embodiments with at least three catheters, one catheter may be used to deliver a saline or glucose solution to the patient.

Further, it is contemplated that the dimensions of catheter assembly 10 may be configured and adapted for applications other than hemodialysis. For example, by increasing or decreasing the catheter size and/or number of catheters and/or lumens or size of lumens in the catheter assembly 10, catheter assembly 10 may be beneficially used for other medical applications in which fluids are introduced into and/or removed from a patient.

Catheters 12, 14 (and/or 16) preferably are made from a material or materials that is or are flexible, durable, soft, and easily conformable to the shape of the area to be catheterized and/or the subcutaneous area, and that minimize risk of harm to blood vessel 44 walls or other tissues of the patient. If catheter assembly 10 is used for hemodialysis applications, it preferably is formed of a relatively softer material that has a hardness of at most about 85-A on a Shore durometer scale. Also, first catheter 12, which may be subjected to negative pressures, may be formed from a harder material (for example 93-A on a Shore durometer scale) than second catheter 14, which may be subjected to positive pressures and which may be formed from a softer material (e.g. 85-A on a Shore durometer scale). For human applications, the materials of manufacture should be non-toxic and non-allergenic.

Catheters 12, 14 (and/and/or 16) can be made of biocompatible plastics and elastomers. Such biocompatible plastics include materials such as, for example, polyethylene, homopolymers and copolymers of vinyl acetate such as ethylene vinyl acetate copolymer, polyvinylchlorides, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyurethanes, polyvinylpyrrolidone, 2-pyrrolidone, polyacrylonitrile butadiene, polycarbonates, polyamides, fluoropolymers such as homopolymers and copolymers of polytetrafluoroethylene and polyvinyl fluoride, polystyrenes, homopolymers and copolymers of styrene acrylonitrile, cellulose acetate, homopolymers and copolymers of acrylonitrile butadiene styrene, polymethylpentene, polysulfones, polyesters, polyimides, polyisobutylene, polymethylstyrene and other similar or equivalent compounds known to those of ordinary skilled in the art. Such biocompatible elastomers include medical grade silicone rubbers, polyvinyl chloride elastomers, polyolefin homopolymeric and copolymeric elastomers, urethane-based elastomers, and natural rubber or other synthetic rubbers. One of ordinary skill in the art can select a biocompatible material suitable for manufacturing catheter assembly 10 without undue experimentation.

In addition to the degradable material 40, catheter assembly 10 may have many of the features present on conventional catheters. For example, catheter assembly 10 can have a radioplaque stripe for aiding in the locating of catheter assembly 10 by a fluoroscope. For another example, catheter 10 can have a fabric cuff that is spaced accordingly to allow for optimal and/or long-term stabilization of catheter assembly 10 in its indwelling position. For another example, catheter assembly 10 can have pair of clamps 26 that can be used to secure or close off catheters before and after a procedure. For another example, catheters 12, 14 (and/or 16) of catheter assembly 10 can have a plurality of apertures is arranged circumferentially around each of the distal ends of the catheters, which function to help prevent stenosis by minimizing the vibratory movement of the distal end regions of catheters 12, 14, (and/or 16) by equalizing the disturbances of intake and return flow. Other features for optimal utilization of catheter assembly 10 are known to those with skill in the art and can be placed on catheter assembly 10 without undue experimentation.

Although embodiments of the present invention are generally disclosed in the context of hemodialysis catheters, it is understood that such embodiments can be applied to other catheters that are used for procedures that require that fluid, blood, medicated solutions, or other solutions to be removed from and/or introduced into a patient. Such procedures include, but are not limited to, hemodialysis, perfusion, chemotherapy, and plasmapheresis.

The above detailed description of the preferred embodiments, examples, and the appended figures are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

1. A multiple lumen catheter assembly comprising:
a) a first catheter having a proximal end and a distal end;
b) a second catheter having a proximal end and a distal end;
c) a degradable material between at least a portion of the first catheter and the second catheter maintaining at least a portion of the first and the second catheter together in an unreleased and unseparated configuration;
whereby the degradable material degrades as fluid contacts the material thereby allowing the distal ends of the first catheter and second catheter to release and separate from each other.

2. The catheter assembly as claimed in claim 1, wherein a portion the first catheter and the second catheter are time-released from each other.

3. The catheter assembly as claimed in claim 1, wherein the degradable material is selected from the group consisting of bioerodable material, biodegradable material, and bioabsorable material.

4. The catheter assembly as claimed in claim 1, wherein the degradable material is selected from the group consisting of collagen, gelatin, elastin, laminin, fribrinogen, fribronectin, vitronectin, dextrose, sucrose, glucose, and fructose.

5. The catheter assembly as claimed in claim 1, wherein the degradable material is selected from the group consisting of polyglycolic acid (PGA), poly-gylcolic and poly-L-lactic acid copolymers, polycaprolactones, polyhydroxybutyrate and hydroxyvalerate copolymers, polycarbonates, poly-L-lactides, polydioxanones, and polyanhydrides.

6. The catheter assembly as claimed in claim 1, wherein the hardness of the first catheter is greater than the hardness of the second catheter.

7. The catheter assembly as claimed in claim 1, further comprising a third catheter having a proximal end and a distal end.

8. The catheter assembly as claimed in claim 5, wherein a portion of the degradable material resides between at least a portion of the outer surface of first catheter and the third catheter thereby allowing the distal ends of the first catheter and the third catheter to release and separate from each other.

9. The catheter assembly as claimed in claim 6, wherein a portion of the first catheter and the third catheter are time-released from each other.

10. The catheter assembly as claimed in claim 1, wherein the first catheter and the second catheter are approximately parallel to each other.

11. The catheter assembly as claimed in claim 1, wherein the lumens of the catheters have a generally semi-circular cross-section.

12. The catheter assembly as claimed in claim 1, wherein one of the catheters is shorter than the other catheter.

13. The catheter assembly as claimed in claim 1, wherein a plurality of apertures is arranged circumferentially around each of the distal ends of the first and the second catheters.

14. The catheter assembly as claimed in claim 1, wherein the degradable material comprises dextrose.

15. A multiple lumen catheter assembly comprising:
a) a first catheter having a harder durometer;
b) a second catheter having a softer durometer; and
c) a degradable material between at least a portion along distal ends of the first and second catheter.

16. The catheter assembly as claimed in claim 15, whereby the degradable material degrades as fluid contacts the material thereby allowing the distal ends of the first and second catheters to release and separate from each other

17. The catheter assembly as claimed in claim 15, wherein the first lumen and the second lumen are approximately parallel.

18. The catheter assembly as claimed in claim 15, further comprising a third catheter.

19. The catheter assembly as claimed in claim 15, wherein the degradable material is selected from the group consisting of bioerodable polymers, biodegradable polymers, and bioabsorable polymers.

20. The catheter assembly as claimed in claim 15, wherein the degradable material is selected from the group consisting of collagen, gelatin, elastin, laminin, fribrinogen, fribronectin, vitronectin, dextrose, sucrose, glucose, and fructose.

21. The catheter assembly as claimed in claim 15, wherein the degradable material is selected from the group consisting of polyglycolic acid (PGA), poly-gylcolic and poly-L-lactic acid copolymers, polycaprolactones, polyhydroxybutyrate and hydroxyvalerate copolymers, polycarbonates, poly-L-factides, polydioxanones, and polyanhydrides

22. A catheter assembly having at least two catheters each having a length, a distal end and a proximal end, a degradable material between walls of at least two of the at least two catheters, wherein the catheters are connected to each other at their proximal ends and releasably connected to each other along a portion between their lengths between their proximal ends and their distal ends.

23. The catheter assembly as claimed in claim 22, wherein the distal ends of at least two of the catheters are time-released from each other by degradation of the degradable material.

24. The catheter assembly as claimed in claim 22, wherein the degradable material is selected from the group consisting of bioerodable material, biodegradable material, and bioabsorable material.

25. The catheter assembly as claimed in claim 22, wherein the degradable material is selected from the group consisting of collagen, gelatin, elastin, laminin, fribrinogen, fribronectin, vitronectin, dextrose, sucrose, glucose, and fructose.

26. The catheter assembly as claimed in claim 22, wherein the degradable material is selected from the group consisting of polyglycolic acid (PGA), poly-gylcolic and poly-L-lactic acid copolymers, polycaprolactones, polyhydroxybutyrate and hydroxyvalerate copolymers, polycarbonates, poly-L-lactides, polydioxanones, and polyanhydrides

27. The catheter assembly as claimed in claim 22, wherein a portion of degradable material resides between outer surfaces of first catheter and the second catheter so to time release the first catheter and the second catheter from each other.

28. The catheter assembly as claimed in claim 22, wherein the catheter assembly has three catheters.
